(19) United States
(12) Patent Application Publication
(10) Pub. No.: US 2006/0153889 A1
(43) Pub. Date: Jul. 13, 2006

(54) Discontinuous surface coating for particles

(76) Inventors: Francis Martin Friel, Westbury, NY (US); Wilson An-Tuen Lee, Hauppauge, NY (US); Aya Shidara, Forest Hills, NY (US); Clara G. Mercado, Saddle River, NJ (US); Eric John Leaver, Huntington Station, NY (US)

Correspondence Address: THE ESTEE LAUDER COS, INC, 125 PINELAWN ROAD, MELVILLE, NY, 11747, US

(21) Appl. No.: 11/324,943

(22) Filed: Jan. 4, 2006

Related U.S. Application Data
(60) Provisional application No. 60/642,721, filed on Jan. 10, 2005.

(51) Int. Cl. A61K 8/29

(52) U.S. Cl. 424/401; 424/63

(57) ABSTRACT

The present invention relates to particle compositions comprising a core particle that is partially coated with a first hydrophilic deposit and a hydrophobic polymeric finish, contains a small amount of water, and a compatible active present in the deposit, the finish or both. Additional alternating deposits, hydrophobic, hydrophilic or both, can be present between the first hydrophilic deposit and the hydrophobic polymeric finish. Similarly, additional compatible actives can be present in one or more of the additional alternating deposits. The invention also relates to methods of preparing the particle compositions.

This application claims benefit of provisional application No. 60/642121, filed on Jan. 10, 2005.

### FIELD OF THE INVENTION

The present invention relates to a discontinuous coating for particulate surfaces. In particular, the invention relates to particle surfaces which have been discontinuously coated so as to be useful in delivering to the skin surface with various actives present therein.

### BACKGROUND OF THE INVENTION

The use of coatings on the surfaces of particles goes back more than forty years in the cosmetic industry. Such coatings are widely used to encapsulate tablets so that they are completely and evenly coated with a coating material. The benefits of a coated tablet include the ability upon degradation of the coating to absorb materials from an environment; to adsorb materials from an environment; or to release materials such as active agents disposed in a matrix of the coating into an environment. Thus, it is known that a coating can possess porosity, such as with a zeolite, and therefore does not require release in order to render absorption or release of a material into or out of the matrix of the coating. In cases such as these, very high selectivity can be obtained by using properly tuned pore characteristics.

The surface treatment of pigments has also been used to improve the ability of incorporating them into cosmetic formulations. For example, pigments coated with different types of silicones are commercially available and when used as cosmetic pigments in formulations the coating facilitates the incorporation of the pigment into hydrophobic formulations whereas the untreated pigment would generally have little affinity. Some pigments arc coated with fluorocarbon polymers to improve their adhesive power while also forming a film upon application. Other pigments are coated with natural polymers such as proteins, for example collagen. However, these types of coatings are not favorable for adhesion of the pigment to the skin and can experience manufacturing difficulties, especially in make-up formulations. The advantage of this type of coating is the ability to introduce molecules or macromolecules such as proteins into formulations even though they are generally anhydrous or have a low water content.

There have been many efforts to create delivery systems and time release mechanisms for actives. For example, actives can be encapsulated to protect the active substance and then to permit its release in a controlled fashion. The actives arc protected underneath a layer(s) of an encapsulation coating. A commonly used material for an encapsulation coating is silicone polymers. Silicone polymers are widely used because they possess two properties of biocompatibility and permeability to gases and small molecules. The process of encapsulating actives is generally by successive film-coating. This process is effective from a technical standpoint and involves at least one stage of spraying a film-forming composition containing an organic polymer in solution in an logic solvent Or in the form of an aqueous emulsion or dispersion followed by at least one drying operation, to evaporate off the organic solvent and/or water. An example of this type of process is known by the name of “spray coating.” In this process particles to be encapsulated are stirred (fluidized) by a gas stream which also ensures their drying (or the evaporation of the organic solvent and/or water). The film-forming composition is sprayed by one or more nozzles situated in various regions of the reactor depending on the process type employed. Another method of encapsulation is in situ cationic (co)polymerization. The object of these types of coating processes is to completely coat the underlying core active material.

It is known further to have a combination of encapsulated particles and unencapsulated particles or with particles that have been encapsulated differently. The use of silicone polymers in-general unfortunately are unsuited for the process of encapsulation using spraying/drying. With the present invention, the disadvantages and increased costs of having to entirely encapsulate an active with polymers is overcome. Sousing advantages are found by depositing multiple actives and materials on the surface of a core particle without the necessity of completely coating the particle surface.

### SUMMARY OF THE INVENTION

The present invention provides a composition for immediate and timed release of actives present in a partial coating on the surface of a core particle. The partial coating is a discontinuous deposit of a first hydrophilic deposit and a hydrophobic polymeric finish and comprises water in an amount of between about 0.01 and 25.00 percent by weight of the particle. The hydrophobic polymeric finish is applied as the last deposit partially coating the core particle surface. Together the discontinuous deposit partially encapsulates the particle. The deposit and the finish partially coat the particle surface. A compatible active is present in the first hydrophilic deposit, the hydrophobic polymeric finish, or both. The method for preparing the particles of the present invention comprises the steps of preparing the first hydrophilic deposit and the hydrophobic polymeric finish, and adding the compatible active to the desired deposit, finish or both. The particles are partially coated with the first hydrophilic deposit, and therefore, the hydrophilic deposit is the first deposit. After the particles are partially coated with the first deposit they are dried until between about 0.01 to 25.00 percent by weight of the particle of water remains. Finally, the particles are partially coated with the hydrophobic polymeric finish as the last deposit partially coating the surface of the particle.

### BRIEF DESCRIPTION OF THE DRAWINGS

FIG. 1 illustrates a particle of titanium dioxide partially coated with a hydrophilic deposit and a hydrophobic finish without fluorescence.

FIG. 2 illustrates a particle of titanium dioxide partially coated with a hydrophilic deposit under fluorescence.

FIG. 3 illustrates a particle of titanium dioxide partially coated with a hydrophobic finish under fluorescence.

FIG. 4 illustrates a particle of titanium dioxide, partially coated with a hydrophilic deposit and a hydrophobic finish under fluorescence.

FIG. 5 illustrates a particle of uncoated titanium dioxide without fluorescence.

FIG. 6 illustrates a particle of titanium dioxide completely coated with a hydrophobic finish.

### DETAILED DESCRIPTION OF THE INVENTION

The immediate and time release particle compositions of the present invention comprise particles tat are surface treated with a first hydrophilic deposit and a hydrophobic polymeric finish. The particle compositions of the present invention are not completely coated Together, the hydrophobic polymeric finish and the first hydrophilic deposit, partially cover the surface of the particle. The first hydrophilic deposit and the hydrophobic polymeric finish form a discontinuous deposit affixed either to the particle surface or to each other. The hydrophobic polymeric finish is the last deposit on the surface of the core particle and the first hydrophilic deposit is the first deposit on the particle surface. Preferably, there is at least one compatible active in the hydrophilic deposit.

Optionally, between the first hydrophilic deposit and the hydrophobic polymeric finish, a first hydrophobic deposit can be added. Further, optionally, between the first hydrophobic deposit and the hydrophobic polymeric finish, at least one additional alternating deposit can be added. The additional alternating deposit is a hydrophobic deposit, the hydrophilic deposit, or combinations thereof. The additional alternating deposits are sequentially clad onto part of the surface of the particle. Therefore, between the first hydrophobic deposit and the hydrophobic polymeric finish there can be one or more alternating hydrophobic deposits and hydrophilic deposits. Any of the hydrophilic deposits, namely the first hydrophilic deposit and the additional alternating hydrophilic deposit may be polymeric or non-polymeric. The additional alternating hydrophobic deposit like the hydrophilic deposits may be non-polymeric or polymeric. As a result any deposit or finish applied to the particle surface can partially coat the surface or any of the other deposits previously partially coated on the surface provided that less than the entire surface is coated at the end of the coating process. However, the additional alternating deposits that are adjacent to one another are incompatible. The finally coated particle is partially coated.

A compatible active is present in at least one of the hydrophilic deposits, the additional alternating hydrophobic deposit or the hydrophobic polymeric finish. When there is only a first hydrophilic deposit and a hydrophobic polymeric finish, the compatible active is present in the first hydrophilic deposit the finish, or both. Compatible actives are ones that are suited to the hydrophilic or hydrophobic nature of the deposit or finish. Thus, hydrophilic actives arc compatible with the hydrophilic deposit and hydrophobic actives are compatible with the hydrophobic polymeric finish. The amount of water remaining on the particle after drying is about 0.01 to 25.00 percent by weight of the particle, preferably about 0.05 to 5.00 percent, and most preferably about 1 to 2 percent. It is desired that a small amount of water remain in the presence of the actives. The presence of water enhances the delivery of the actives in the hydrophilic deposit onto the skin surface. While not wishing to be bound by any particular theory, it is believed that an enhanced delivery of actives is achieved by osmotic diffusion.

The discontinuously coated particles provide a delivery system and a time release mechanism for the actives contained in the first hydrophilic deposit and/or the hydrophobic polymeric finish, and optionally, in the additional alternating deposits. Any of the first hydrophobic deposit, the first hydrophilic deposit or the additional alternating deposits can be partially coated with the last deposit of the hydrophobic polymeric finish or any previously applied deposit. The last deposit of the hydrophobic polymeric finish aids in controlling the release of the actives in both the underlying hydrophilic deposits and the hydrophobic deposits that it partially covers. Not all of the deposits will be covered by the last deposit because it only partially covers the underlying deposits. As a result, the previously clad deposits are either uncovered or partially covered by the last deposit of the hydrophobic polymeric component. The partial coating of the hydrophilic deposit and the hydrophobic deposit is chemically non-reactive with the particle as well as with each other. Therefore, the hydrophobic deposit and the hydrophilic deposit adjacent to one another are incompatible with one another. Thus, it is surprising that alternating partial coatings of the hydrophobic deposit and/or the hydrophilic deposit can be partially clad on the core particle. The discontinuity of the deposits facilitates the release of actives in the specific hydrophobic and/or hydrophilic deposit in a range of times from immediate to sustained release over time. Further, the particles of the present invention also have the benefit of protecting the actives from oxidation and any other environmental factors that could cause the active to degrade or be rendered unstable or inactive.

The amount of the compatible active in either the hydrophobic or the hydrophilic deposit, first or additional alternating, partially covering the core particle is about 0.01 to 10.00 percent by weight of the particle composition, and preferably, 0.01 to about 2.00 percent by weight of the particle. The actives of the hydrophilic or the hydrophobic deposit arc randomly either uncovered or partially concealed by one or more subsequently applied deposits of the hydrophobic deposit, or the hydrophilic deposit. While the particle is only partially covered it is possible for underlying deposits to be fully covered by subsequently applied additional alternating deposits and/or the hydrophobic polymeric finish. Thus, not all actives release at the same time in equal amounts. The actives in the hydrophobic or hydrophilic deposit, including the first deposit and the additional, alternating deposit, that are uncovered are one of the first actives to be released, and are therefore, considered to be immediate released actives. With respect to the first hydrophilic deposit, if it contains actives and if the first hydrophilic deposit is coated by subsequent deposits or the hydrophobic polymeric finish, those actives are time released. In the case where there are alternating hydrophobic and hydrophilic deposits, actives in underlying hydrophobic or hydrophilic deposits are released after the actives in any of the overlaying hydrophobic deposits or hydrophilic deposits and are also time released. Therefore, the particles of the present invention are immediate and time released by virtue of each partially coated particle immediately and over time releasing actives contained in the deposits and the finish.

The amount of uncovered or partially concealed actives that is released depends on variables associated with the skin surface to which the compositions of the present invention are applied, the active and the thickness of the partial coating. These variables include, but are not limited to, skin conditions such as pH, proteases, and sebum oil, the thickness of the partially spread deposit and the diffusivity of the active itself. Thus, for example, sebum levels, containing glycerides, free fatty acids, wax esters, squalene cholesterol esters and cholesterol, are related to the release or activation of the active in the last deposit Sebum production from sebaceous glands varies with age and by sex, and therefore, the composition of oils is altered. In addition, the hydrophobic polymeric finish reacts with stratum corneum proteases, for example, human stratum corneum trypsin-like serine protease, chymotypsin-like serine proteases, casein digestible proteases, on the skin to individually tailor the release of actives to the needs of the skin of the individual user. The hydrophobic polymeric finish breaks down and releases any actives within or underlying the finish in the deposits as a result of reacting with the proteases present on the skin surface to which the particles of the present invention arc applied. The thickness of the partially spread deposits are random and are directly proportional to the amount of time that the particle is partially coated. The time release of the actives in the partial deposits inversely relates to the thickness of the partial deposit (the thicker the deposit, the slower the release). This is achieved with the partial encapsulation of the particle with not only the uncovered deposits which provide the immediate release of the active contained therein, but also with the additional alternating deposits and the hydrophobic polymeric finish which provides a varied time release of any of the actives contained therein. Further, certain actives possess skin permeation characteristics. Vitamin C, for example, has been found to permeate rapidly through the skin.

The hydrophilic deposit is an aqueous based media that includes, but is not limited to, water, glycols, glycerin, water-based gums, carbomer/water-based gels, acrylates copolymers, and quaterary amine compounds. By the term “aqueous based media,” it is meant in the present specification that the media can be, for example, a solution, an emulsion, a slurry, or a dispersion. The hydrophilic deposit may or may not contain actives.

The hydrophilic actives If they are present in any of the hydrophilic deposits are simply added to the hydrophilic deposit. The hydrophilic actives include, but are not limited to, water soluble preservatives, carbohydrates, water soluble vitamins, amino acids, and synthetic and natural water soluble extracts. Examples of water soluble extracts include but are not limited to, extracts from, artemia, phytosphingosine, polygons cuspidatun root, yeast such as saccharomyces lysate, thermos themophillus ferment, birch (Betula alba), mimosa tenuiflora (bark) extract fruit, clove, rye, malt, corn, spelt, millet, barley, oat, wheat, sesame, cumin, turmeric, green onion, celery, ginseng, ginger, licorice, carrot, bupleunum root, Ginkgo biloba (gingko), Poeniculi Fructis (fennel), kiwi, berry such as Morus bombycis (mulberry), Gentiana lutea (gentian), algae such as red algae, Arctium lappa (burdock), Salvia officinalis (sage), Lentinus edodes (shiitake mushroom), Perilla frutescens (perilla), Filipendula Multijuga, Fucus vesiculosis (bladderwrack, sea weed), peach kernel, Panax ginseng, carrot, Allium sativum (garlic), Poria cocos (poria), Humulus lupulus (hops), Mutan Cortex (Mouta Bark), Pimpinella major, Lactuca sative (lettuce), Astrgalus membranaceous (astagalus) and Rosmarinus officinalis (rosemary), Primus amygdalus (almond), Althca officinale (althea), aloe, Rosae Fructus (rose fruit, or Rosa multiflora), Scuttelaria baicalensis (Huang qin), Pucrariae Radix (Pueraria Root, or Pueraria lobata), chamomile such as Chamomillae Flos (German chamomile), Gardenia jasminoides (zhii zi, Gardeniae Fructus), Sophora flavescens Alton (Sophorac Radix), chlorella, rice bran, Paeoniae lactiflora (white peony), ziyu (Sanguisorba officinatis, burnet), Morms alba (sang bai pi, mulberry), Clycine max (soybean), Camellia sinensis (tea), Carthami Flos (safflower), Aesculus hippocastanum (horse chestnut), Melissa officinalis (lemon balm) and Coicis Semen (Coix lacrma-jobi var. ma-yuen), Angelica keisulcei, Amica montana (arnica), Foeniculum officinale (fennel), Isodon japonicus Hara (Isodonis Herba), Daucus Carota (carrot), Oryza sativa (rice), Crataegus cuneata (Japanese howthom), Acorns calas (sweet flag), Crataegus oxycantha (howthon), Juniperus colmmunis, Ligusticum wallichii (Chinese lovage), Swvertiae Herba (Swertia Herb), Thymus vulgaris (garden thyme), Citrus reticulata (Citrus unshiu), Capsicum tincture, Angelicae sinensis (angelica), Aurantii Pericarpium (bitter orange peel), Ruscus aculeatus (butchcrls bloom), Vitis vinifera (grape), Tilia japonica (lime), Citrus junos and Rosa canina (rose hip), caffeine, Cinnamomi Cortex (cinmamon bark) and Eriobotrya japonica Lindl. (loquat), Gumbir, Echinacea, Phellodendri Cortex (amur cork tree or Phellodendron amurense), Hypericum perforatum (St. John's wort), Citrus sinensis (orange), Valcriana fauriei Briquet, Arisia capillaris Thunb., Cucumis sativus (cucumber), Geranii Herba (Geranium Herb), Lithospermum erythrorbizon Sieb. et Zucc., Hedera helix, Achillea millefolium (brarrow), Ziziphus jujuba (Chinese dates), Calendula officinalis (pot marigold), Houttuynia cordata (Houttyniae Herba, Houttuynia Herba), Potentilla erecta, Petroselinum crispum (parsley), Parietaria officinialis, Santalum album (sandalwood), Primus persica (peach), Centaurea cyanus (cornflower), Eucalyptus globulus (eucalyptus) and Lavandula angustifolia (lavender), Persea americana (avocado), Aloe vera (aloe), Nasturtium officinalis (watercress), Symphytum offcinale (cornfrey), Asarsuim sieboldii (wild ginger), Xanthoxyum piperitum (Japan pepper), Richmannia glutinosa (di huang), Mentha piperita (eppcrmint), Syzygium aromaticum (clovc), Tussilago farfara (coltsfoot) and Haematoxylum campechianum (logwood); Oolong tea, Cinchona succirubra (Peruvian bark), Betula verucosa (birch) and Glechoma hederacea (ground ivy), milk and royal jelly, honey, cysteine and derivatives thereof, ascorbic acid and derivatives thereof, BHA, BHT, ferulic acid and derivatives thereof, grapeseed extact, pine bark extract horseradish extract, hydroquinones, rosmarinic acid, coffee robusta seed, caffeic acid, tocopherol and derivatives thereof, green tea extract, sodium DNA, sodium ribonucleic acid, octyl, propyl and dodicyl gallates, uric acid and thiodiproprionate derivatives, cysteine and derivatives thereof, ascorbic acid and derivatives thereof, BHIA, BHT, ferulic acid and derivatives thereof, grapeseed extract, pine bark extract, horseradish extract, hydroquinones, rosmarinic acid, coffee robusta seed, caffeic acid, tocopherol and derivatives thereof, green tea extract, sodium DNA, sodium ribonucleic acid, octyl, propyl and dodecyl gallates, uric acid and thiodiproprionate derivatives. Examples of preferred water soluble actives include but are not limited to ascorbic acid and water soluble derivatives such as ascorbyl tocopheryl maleate or aminopropyl ascorbyl phosphate, salicylic acid, nordihydroguaiaretic acid, polygonum cuspidcatur root extract, betula alba (birch) extract, yeast extract (e.g., saccharomyces lysate extract), rosemary extract, soybean extract, artemia extact, thermus thermophillus ferment extract, and coffea robusta seed extract.

The hydrophobic deposit can be polymeric or non-polymeric. Generally speaking, the hydrophobic deposit can be oils, silicones, hydrocarbons, waxes, esters, organic compounds, and resins. The hydrophobic non-polymeric deposit includes but is not limited to, vegetable oils, such as coconut oil, jojoba oil, corn oil, sunflower oil, palm oil, soybean oil, carboxylic acid esters such as isostearyl neopentanoate, cetyl octanoate, cetyl ricinoleate, octyl palmitate, dioctyl malate, coco-dicaprylate/caprate, decyl isosteatate, myristyl myristate, animal oils such as lanolin and lanolin derivatives, tallow, mink oil or cholesterol; glyceryl esters, such as glyceryl stearate, glyceryl dioleate, glyceryl distearte, glyceryl linoleate, glyceryl myristate; non-volatile silicones, such as dimethicone, dimethiconol, phenyl trimethicone, methicone, simehicone; volatile silicones such as cyclomethicone; volatile hydrocarbons such as isododecane; and non-volatile hydrocarbons, such as isoparaffins, squalane, or petrolatum, waxes, ester/organic compounds. As a hydrophobic polymeric deposit or finish, examples include resins such as siloxane resins and acrylate resins (polyrized syntheic thermosetting materials), polymers such as silicones, and specifically film-forming silicone polymers such as dimethicone copolyol, trimethylsiloxysilicate, and polymers having a fluorinated carbon chain, and can include oxazoline-modified organopolysiloxanes, vinyl copolymers polymers consisting of two or more different monomers) containing a polysiloxane macromer, organopolysiloxanes having a sugar residue, alkyl-modified organopolysiloxates, high polymerization organopolysiloxanes, oxazoline-modified organopolysiloxanes, homopolymers of a fluorine-containing vinyl monomer, copolymers containing a fluorine-containing vinyl monomer as a constituent and vinylidene fluorifle-hexafluoroacctone copolymer, fluoroallyl (meth)acrylates, fluoroalkyl (meth)aerylic amide esters, fluoroalkyl vinyl ethers and fluorto-alpha-olefins, of which (meth)acrylates having a fluoroalwyl group (having 6 to 12 carbon atoms). The hydrophobic deposit or finish is present in an amount of about 0.001 to 30 percent by weight of the particle, preferably about 0.005 to 10 percent, more preferably about 0.01 to 5 percent. Film formers are preferred as the finish and trimethylsiloxysilicate is particularly preferred as the finish, and therefore, the present particle compositions have an additional benefit with respect to film forming and waterproofing.

Like the hydrophilic deposit, the compatible active is simply added to the hydrophobic polymeric finish or the hydrophobic deposit, for or additional alternating. To hydrophobic actives include, but are not limited to, oil soluble vitamins, oil soluble extracts, and oil soluble actives such as tocopherol, yeast cell derivatives, tritium vulgare (wheat) germ., microporous cellulose, cholecalciferol, colloidal oatmeal, Peruvean balsam oil, protein hydrolysate, racemic methionine, Vitamin A benzophenone-3, avobetuone, ethylboxyl dimethyl PABA, methoxycinnamates, ethylbexyl salicylate, homosalate, 3-benzylidene camphor, ethythexyl triazone, isopropylbenryl salicylate, 4-methylbenzylidene camphor, octocrytene, coated particles of zinc oxide and titanium dioxide treated to be oil soluble, and other particles that are treated to be oil soluble can be present in the hydrophobic deposit.

The particle useful in the present invention can be any water-insoluble or water-soluble, porous or non-porous solid material that is capable of being treated to permit a discontinuous deposit onto its surface. The method of treating the core particle can be any known process for applying coatings onto the surface of a particle including but not limited to jet milling, and fluid bed granulizing. Specific examples of the water-insoluble core particle include, but are not limited to, mineral pigments and mineral fillers such as, for example, talc, kaolin, mica, bismuth oxychloride, chrorium hydroxide, barium sulfate, polynethylmetb.acrylate (PMMA), baron nitride, nylon beads, silica, silica beads, lakes such as aluminum lake or calcium lake, metal oxides such as black, yellow or blue iron oxide, chromium oxide, zinc oxide or titanium dioxide, plastic powder such as BPID 500, physical and chemical sun filters, salts, sugars and other like organic and inorganic powders and particles. Specific examples of water-soluble core particles include, but are not limited to, salts such as, for example, sodium chloride, magnesium chloride, magnesium sulfate and calcium sulfate, potassium chloride, hydrated sodium sulfate, and magnesium bromide; and sugars such as, for example, sucrose, dextrose, fructose, levulose, glucose, lactose, maltose, and galactose. Preferably, the particles are nano-sized meaning that they have a particle size of about 1 to 20 microns; preferably, 2 to 15 microns, and more preferably, an average of about 6 to 10 microns. After treating the particle to affix the first deposit and the second deposit to the surface of the particle, the treated core particles are about 10 nanometers to 300 microns, preferably in size. The particles can be any shape irregular, spherical, plates. However, preferably they are irregular in shape and are not spherical. The core particles comprise about 75 to 95 percent by weight of the composition, preferably, about 80 to 95 percent.

To prepare the composition of the present invention, the first hydrophilic deposit is randomly coated onto part of the surface of the particle. Any method known in the art of coating a particle can be used to partially coat the particles such as, for example, a fluid bed granulator (Glatt OPGC-1, Glatt Air Techniques, Ramsey, N.J.). When using a fluid bed granulator, a pre determined amount of particles, for example between 75 and 95 percent by weight of the particle, are added to the granulator and fluidized at a pressure range of about 0.2 to 1.0 bar pressure (inlet air flow) and at an inlet air temperature (IAT) set point of about 10° C. The first hydrophilic deposit is fed into the granulator at a feed rate of about 10 to 15 grams per minute for, as an example, an 800 gram batch and is top sprayed onto the particles at a pressure range of about 1.0 to 5.0 bar atomization pressure (i.e., air pressure that atomizes liquids) and at an IAT of about 60° C. Heat is applied after the first hydrophilic deposit is coated onto the particle for about 10 to 30 minutes to allow the water content to reach about 1 to 2 percent of the particle. After the drying step is complete, the temperature of the partially coated particle is reduced to about 48° C. and fluidization is ccased. To partially coat the particle with the first hydrophobic deposit, or the additional alternating hydrophobic or hydrophilic deposits, the step of fluidization is repeated using the pressure range of about 0.2 to 1.0 bar pressure and 10° C. IAT. If one of the additional deposits is a volatile hydrophilic deposit, another drying step can be applied. The last deposit of the hydrophobic polymeric finish is applied using the same pressure range and temperature for the previous fluidization steps.

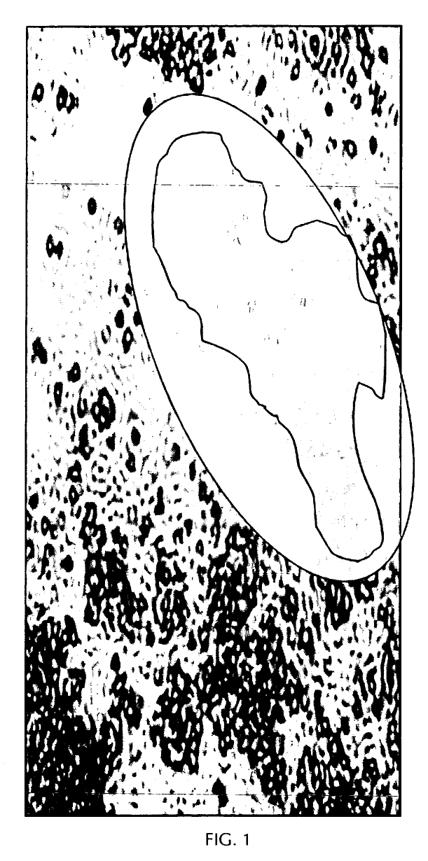

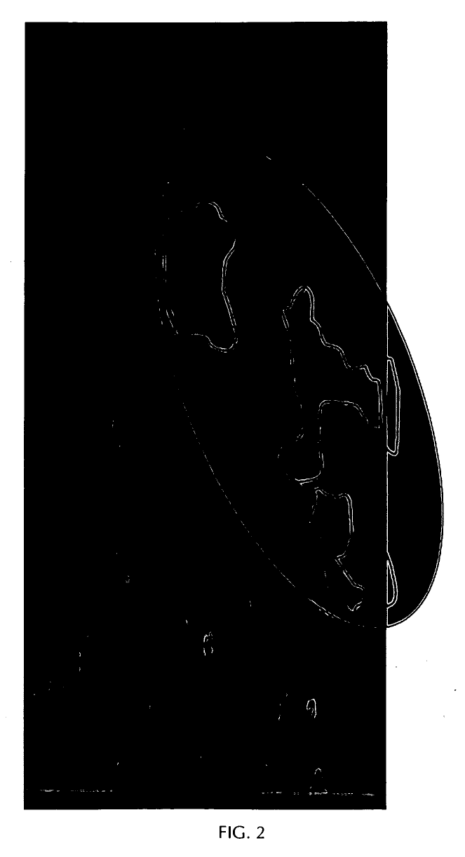

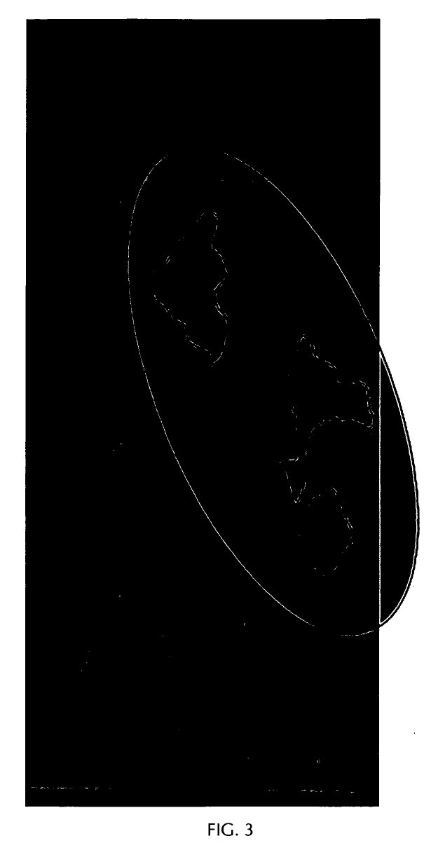

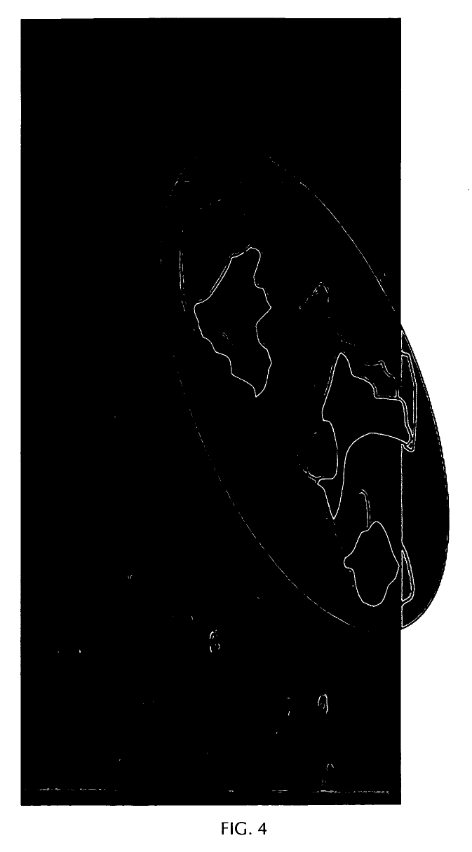

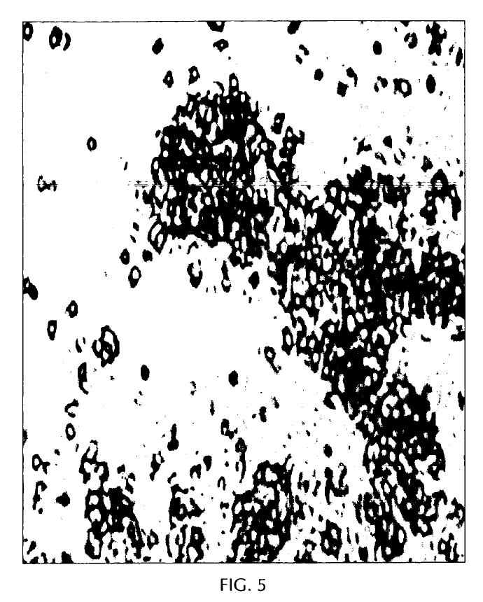

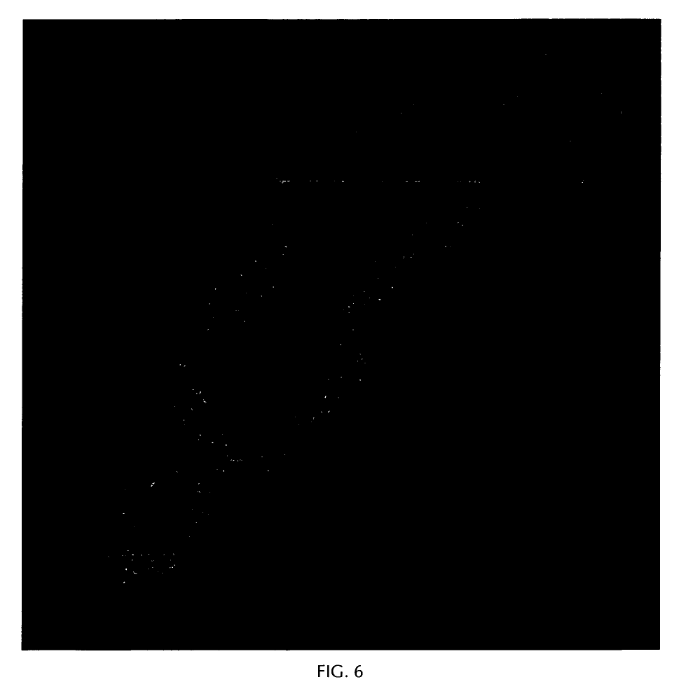

The partial coverage of the particles by the method of the present invention is depicted in the FIGS. 1 through 3. In FIG. 1, the present invention of the partially coated particle is illustrated wherein titanium dioxide is partially coated wit the hydrophilic deposit and the hydrophobic finish. Within the oval of FIG. 1, as well as the other figures, an outline shows the titanium dioxide particle without the application of fluorescent light and it is not possible to differentiate between the hydrophobic and hydrophilic partially coated areas. To further demonstrate this point, in FIG. 5 a completely uncoated titanium dioxide particle is shown and it looks very similar to FIG. 1. In order to highlight the two different deposits and finish, a fluorescence technique is employed, In FIG. 2, the hydrophilic deposit partially coating the titanium dioxide is shown by fluorescence. There are three outlined areas within the outline of the titanium dioxide (the outline shown in FIG. 1). Each of the three outlined areas depicts areas of the titanium dioxide that are partially coated with the hydrophilic deposit. Next, in FIG. 3, also under fluorescence, the titanium dioxide is partially coated with the hydrophobic finish and three outlined areas within the titanium dioxide outline indicate the partial coating of the hydrophobic finish. To demonstrate the ability to adequately view the hydrophobic and the hydrophilic portions of the particle that are partially coated, in FIG. 6, a particle is completely coated with the hydrophobic finish and illustrates that the entire coverage of the particle can be observed. Finally, in FIG. 4, the titanium dioxide is partially coated with both the hydrophilic deposit and the hydrophobic finish is shown under fluorescence. The three outlined areas of the partial coating of the hydrophilic deposit and the three outlined areas of the partial coating of the hydrophobic finish are superimposed within the outline of the titanium dioxide to show partial coating of the particle by both the deposit and the finish. From this illustration it can be seen that there are areas of the deposit that are both uncovered and partially covered by the finish. Further, it can be seen that the particle is partially covered.

The partially coated particles of the present invention can be added directly thereto, any type of cosmetic composition or formulation. The discontinuously coated composition prepared as described above is ready to be added to a cosmetically acceptable carrier or base of choice. The composition can be used in any cosmetic base in which a pigment would be necessary or desirable, using standard methodology. For example, the pigment composition can be incorporated into wax-based products, such as lipsticks and lip glosses, fluid products (either aqueous or non-aqueous) such as foundations, cream eye shadows and blushes, and lotion and cream treatment, and powder products, such as face powder, powder blushes and powder eye shadows, nail products, skin treatment products. When topically applied to the skin via a cosmetic composition actives are released from the partial coating on the particle surface.

The invention is for illustrated in the following non-limiting examples.

### EXAMPLES

Particles with First Hydrophilic Deposit and Hydrophobic Polymeric Finish

| Ingredient | Percent |
|---|---|
| Phase I | |
| Titanium dioxide | 92.0 |
| Phase II | |
| Water | 4.0 |
| Resveratrol | 0.4 |
| Yeast extract | 0.3 |
| Rosemary extract | 0.1 |
| White birch extract | 0.2 |
| Phytosphingosine | 0.1 |
| Ascorbyl tocopheryl maleate | 1.5 |
| Soybean extract | 0.1 |
| Artemia extract | 0.1 |
| Thermus thermophillus | 0.1 |
| Coffee seed extract | 0.1 |
| Phase III | |
| Dimethicone | 0.5 |
| Trimethylsiloxysilicate | 0.5 |

The titanium dioxide of phase I is added to a fluid bed granulator (Glatt GPGC-1, manufacturer city, state) and it is fluidized using 0.4 bar pressure (inlet air flow) and 10° C. inlet air temperature (“IAT”) set point. The first hydrophilic deposit of water soluble actives of Phase II are top sprayed onto part of the surface of the titanium dioxide at 2.5 bar atomization pressure, pump set to 5 rpm. After water soluble actives are sprayed onto part of the surface, the titanium dioxide treated with a discontinuous coating of a first deposit of actives is fluidized at 0.4 bar pressure (inlet air flow) and 60° C. IAT set point and dried for about 20 minutes. Once drying is complete the temperature of the titanium dioxide treated with the first deposit is brought down to about 48° C. and fluidization is ceased. Fluidization is repeated using 0.4 bar pressure (inlet air flow) and 100° C. IAT set point. The trimethylsiloxysilicate (the hydrophobic polymeric finish) of Phase III is sprayed onto part of the surface of the titanium dioxide treated with the first deposit. The final particle is partially coated with actives for timed release and is ready to add to a cosmetic composition

Particles with First Hydrophilic Deposit, First Hydrophobic Deposit and Hydrophobic Polymeric Finish

| Ingredient | Percent |
|---|---|
| Phase I | |
| Titanium dioxide | 88.0 |
| Mica | 2.0 |
| Phase II | |
| Water | 4.0 |
| Mimosa tenuiflora extract | 0.6 |
| Butylene glycol | 2.4 |
| Aminopropyl ascorbyl phsophate | 0.4 |
| Water soluble preservative | 0.1 |
| Phase III | |
| Dimethicone | 1.0 |
| Wheat germ extract | 0.5 |
| Phase IV | |
| Dimethicone | 0.5 |
| Trimethylsiloxysllicate | 0.5 |

The titanium dioxide and mica of phase I is added to a fluid bed granulator (Glatt OPGC-1, manufacturer city, state) and it is fluidized using 0.4 bar pressure (inlet air flow) and 1000 inlet air temperature (“IAT”) set point. The first hydrophilic deposit of water soluble actives of Phase III are top sprayed onto part of the surface of the titanium dioxide and mica at 2.5 bar atomization pressure, pump set to 5 rpm. After water soluble actives are sprayed onto part of the surface, the titanium dioxide and mica treated with a discontinuous coating of a first hydrophilic, deposit of actives is fluidized at 0.4 bar pressure (inlet air flow) and 6000 IAT set point and dried for about 20 minutes. Once drying is complete the temperature of the titanium dioxide and mica treated with the first hydrophobic deposit is brought down to about 48° C. and fluidization is ceased. Fluidization is repeated using 0.4 bar presser (inlet air flow) and 10° C. IAT set point. The first hydrophobic deposit of Phase In is sprayed onto part of the surface of the titanium dioxide treated with the first hydrophilic deposit. Following the application of the first hydrophobic deposit of Phase III, the methylsiloxysilicate (the hydrophobic polymeric finish) of Phase IV is sprayed onto pant of the surface of the titanium dioxide and mica treated with the first hydrophilic and hydrophobic deposits. The final particle is partially coated with actives for timed release and ready to add to a cosmetic composition.

Study of Partial Coating

The partial coating of the immediate and time release particles of the present invention are studied by analyzing fluorescent pictures of the particle with the hydrophilic deposit and the hydrophobic finish. Double labels are prepared to project two images, object of the hydrophilic deposit and the other of tie hydrophobic finish, from an optical plan within the sample of partially coated titanium dioxide. The particles are coated with a hydrophilic solution that has 2 g of green fluorescent BODIPY™ FL C₁₆ added to 200 g solution to label the hydrophilic deposit, and a hydrophobic solution that has 2 g of red Fluorescent β-BODIPY™ 530/550 C₅/HPC added to a 250 g solution to label the hydrophobic finish according to the present invention. The prepared sample is shielded light sources. A 1 to 4 g sample is placed and flattened onto a micro slide and examined under a confocal microscope. The full image of the sample is obtained by moving an image point across the sample using mirror scanners. The emitted/reflective light passing through the detector pinhole is transformed to electrical signals by a photo multiplier and displayed on a computer monitor screen. To obtain image of the hydrophilic deposit on the surface of the particle, regions of 560 nm wavelength are used and the image of the hydrophilic deposits are saved in a data file. On the same area of the particle, 630 nm wavelength is used to show the hydrophobic finish on the particle spice and is saved in a data file. The total discontinuous deposit on the particle is calculated mathematically. A weight method is applied to demonstrate the partial coating of the particle. A photocopy of the pictures is taken of the particles in FIG. 2 (particle partially coated with hydrophilic deposit), FIG. 3 (particle partially coated with hydrophobic finish) and FIG. 4 (particle partially coated by both the hydrophilic deposit and the hydrophobic finish). Each picture of the entire particle is weighed in grams on a microscope, Sartorius CP64, Gottingen, Germany.

For FIG. 2 of the partial coating of hydrophilic deposit, the three outlined hydrophilic areas are cut out of the picture and weighed. This weight symbolizes the portion of the particle partially covered by the hydrophilic deposit. Next the remaining area of the particle is weighed and this weight represents the area of the particle uncovered by the hydrophilic deposit The FIG. 2 data is: entire particle is 0.225 g, area of the particle partially covered by the hydrophilic deposit is 0.0895 g, and area of the particle not covered by the hydrophilic deposit is 0.135 g. The same process is applied for the hydrophobic finish and the FIG. 3 data is entire particle is 0.2235 g, area of the particle partially covered by the hydrophobic finish is 0.0723 g, and area of the particle not covered by the hydrophilic deposit is 0.152 g; and the same process is applied for both the hydrophobic finish and the hydrophilic deposit partially covering the particle in FIG. 4 and its data is: entire particle is 0.2301 g, area of the particle partially covered by both is 0.0324 g, and area of the particle not covered by the hydrophilic deposit is 0.1976 g. As a result of this study it is found that the particle is uncovered by 42 percent, and that the particle is covered 26 percent by only the hydrophilic deposit, 18 percent by only the

Hydrophobic deposit, and covered 14 percent by both the deposit and the finish. Thus, the surface of the particle is partially coated with the deposit and the finish.

What we claim is:

1. An immediate and time release particle comprising water in an amount between about 0.01 and 25.00 percent by weight of the particle, a surface partially coated with a first hydrophilic deposit and a hydrophobic polymeric finish, and a compatible active present in the first hydrophilic deposit, the hydrophobic polymeric finish, or both the deposit and the finish.

2. The particle of claim 1 wherein the hydrophilic deposit comprises the compatible active.

3. The particle of claim 1 further comprising a first hydrophobic deposit between the first hydrophilic deposit and the hydrophobic polymeric finish.

4. The particle of claim 3 further comprising at least one additional alternating deposit selected from the group consisting of a hydrophobic deposit, a hydrophilic deposit, and a combination thereof between the first hydrophilic deposit and the hydrophobic polymeric finish.

5. The particle of claim 4 wherein the additional alternating deposit comprises at least one compatible active.

6. The particle of claim 1 wherein the hydrophobic polymeric finish comprises the compatible active.

7. The particle of claim 1 wherein the hydrophobic polymeric finish is a film former.

8. The particle of claim 7 wherein the film former is trimethylsiloxysilicate.

9. The particle of claim 1 wherein the water content is between about 0.05 and 5.00 percent by weight of the particle.

10. The particle of claim 1 wherein the hydrophobic deposit is selected from the group consisting of oils, silicones, hydrocarbons, waxes, esters, organic compounds, and resins.

11. The particle of claim 4 wherein additional alternating deposits adjacent to one another are incompatible.

12. The particle of claim 1 wherein the hydrophilic deposit is selected from the group consisting of water, glycols, glycerin, water-based gums, carbomer/water-based gels, acrylates copolymers, and quaternary amine compounds.

13. The particle of claim 1 wherein the hydrophilic active is selected from the group consisting of water soluble vitamins, water soluble preservatives, amino acids, and water soluble extracts.

14. The particle of claim 1 wherein the core particle is selected from the group consisting of talc, kaolin, mica, bismuth oxychloride, chromium hydroxide, barium sulfate, polynethylmetb.acrylate (PMMA), boron nitride, nylon beads, silica, silica beads, lakes such as aluminum lake or calcium lake, metal oxides such as black, yellow or blue iron oxide, chromium oxide, zinc oxide or titanium dioxide, plastic powder such as BPD 500, physical and chemical sun filters, salts, sugars and other like organic and inorganic powders and particles.

15. The particle of claim 14 wherein the particle is titanium dioxide.

16. A method for preparing an immediate and time release particle having a surface partially coated comprising the steps of (a) preparing a first hydrophilic deposit and a hydrophobic polymeric finish; (b) adding a compatible active to the first hydrophilic deposit, the hydrophobic polymeric finish, or both of the deposit and the finish; (c) partially coating the surface with the first hydrophilic deposit; (d) drying the particles until between about 0.01 to 25.00 percent by weight of the particle of water remains; and (e) partially coating the surface with the hydrophobic polymeric finish.

17. The method of claim 16 further comprising the step of partially coating the surface with a first hydrophobic deposit between the step of drying the particles and the step of partially coating the surface with the hydrophobic polymeric finish.

18. The method of claim 17 further comprising the step of partially coating the surface with additional alternating deposits selected from the group consisting of a hydrophilic deposit, a hydrophobic deposit, and a combination thereof, between the step of partially coating the surface with the first hydrophobic deposit and the step of partially coating the surface with the hydrophobic polymeric finish.

19. The method of claim 16 in which the hydrophobic polymeric finish is trimethylsiloxysilicate.

20. The method of claim 17 in which the first hydrophobic deposit is dimethicone.

21. The method of claim 16 in which the first hydrophilic deposit is selected from the group consisting of water, glycols, glycerin, water-based gums, carbomer/water-based gels, acrylates copolymers, quaternary amine compounds, and carbohydrates.

22. The method of claim 16 in which the particle is selected from the group consisting of titanium dioxide, oxides (iron and zinc), mica, silica, bismuth oxychloride, PMMA (polymethylmethacrylate), lakes (aluminum and calcium), talc, plastic powders (BPD 500), salts, sugars, organic pigments, inorganic pigments, organic powders, and inorganic powders.

23. The method of claim 16 further comprising the step of adding the controlled release particles to a cosmetically acceptable carrier.

24. The method of claim 23 further comprising the step of topically applying the particle to the skin containing stratum corneum proteases.

25. The method of releasing actives from partially coated particles to the surface of the skin containing stratum corneum proteases comprising the step of topically applying the particles of claim 1 to the skin.

26. The method of releasing actives from partially coated particles to the surface of the skin containing stratum corneum proteases comprising the step of topically applying the particles of claim 3 to the skin.

27. The method of releasing actives from partially coated particles to the surface of the skin containing stratum corneum proteases comprising the step of topically applying the particles of claim 4 to the skin.

28. A cosmetic composition comprising the particles made according to claim 16.

29. A cosmetic composition comprising the particles made according to claim 17.

30. A cosmetic composition comprising the particles made according to claim 18.
